(19) United States
(12) Plant Patent Application Publication
(10) Pub. No.: US 2013/0318664 P1
(43) Pub. Date: Nov. 28, 2013

(54) Raspberry plant - Lagorai Plus cultivar

(50) ;

(71) Applicant: Aldo Telch, Faver (IT)

(72) Inventor: Aldo Telch, Faver (IT)

(21) Appl. No.: 13/815,342

(22) Filed: Feb. 22, 2013

(30) Foreign Application Priority Data
May 25, 2012 (IT) CPVO 201200708

(51) Int. Cl. A01H 5/00

(52) U.S. Cl. PLT/204

(57) ABSTRACT

The raspberry cultivar Lagorai Plus was obtained by crossing of two unknown hybrid raspberry at the Val di Cembra, Faver, in Trentino Region of Italy. The new cultivar is a primocane variety producing two crops per year, wherein the fruits ripen on current season's cane in very late autumn and on previous season's cane in mid-summer. The production of autumn is negligible when in summer is very high. The resulting fruits are conical and medium red in coloration.

### GENUS AND SPECIES

Rubus idaeus L.

### VARIETY DENOMINATION

“Lagorai Plus”

### BACKGROUND OF THE NEW VARIETY

A new and distinct cultivar of red raspberry (Rubus idaeus L.) hereinafter referred to by the cultivar denomination “Lagorai Plus” is disclosed. The new variety “Lagorai Plus” was obtained from the cross of unknown individual raspberry plants by free pollination.

The crossing for the new cultivar occurred on year 2003 in Val di Cembra (Faver, Trento—Italy) and it has been subject to selection on year 2005 and then tested since introduction on year 2007. The characteristics of the new cultivar have been found stable and have been transmitted without change through succeeding (more than 6) asexual propagations.

### SUMMARY OF THE INVENTION

The most outstanding and distinguishing characteristics of the “Lagorai Plus” cultivar are hereunder reported.

Primocane raspberry plant, but is more interesting growing the variety as floricane;

Good fruit colour and flavour;

Large and firm berries.

The new cultivar can be propagated by: the use of suckers produced in a spawn-bed, root cutting and use of tissue culture techniques. The root cutting is to be preferred.

### BRIEF DESCRIPTION OF THE PHOTOGRAPHS

The new red raspberry cultivar “Lagorai Plus” is illustrated by the accompanying photographs showing typical specimens of the new variety in colour as true as reasonably possible to make the same colour illustration of this character by conventional photographic procedures. The photographs are of plants that are two years old.

FIG. 1 shows the fruit size, shape and colour at ripening.

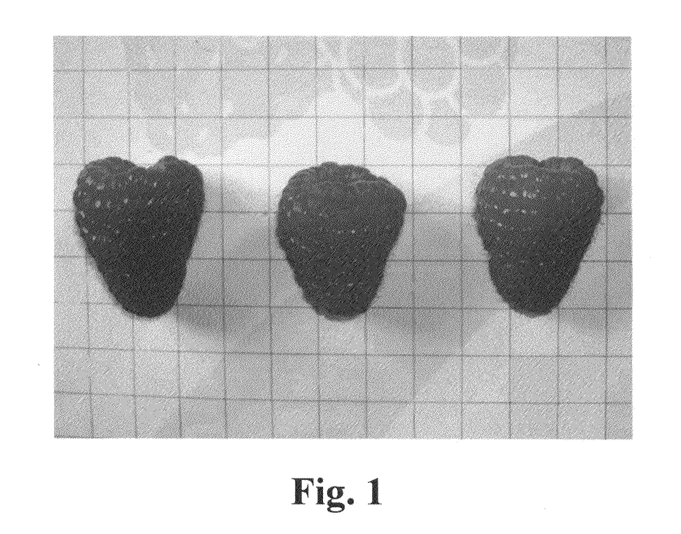

FIG. 2 a view of a raspberry plants showing, in particular, typical distribution of fruit.

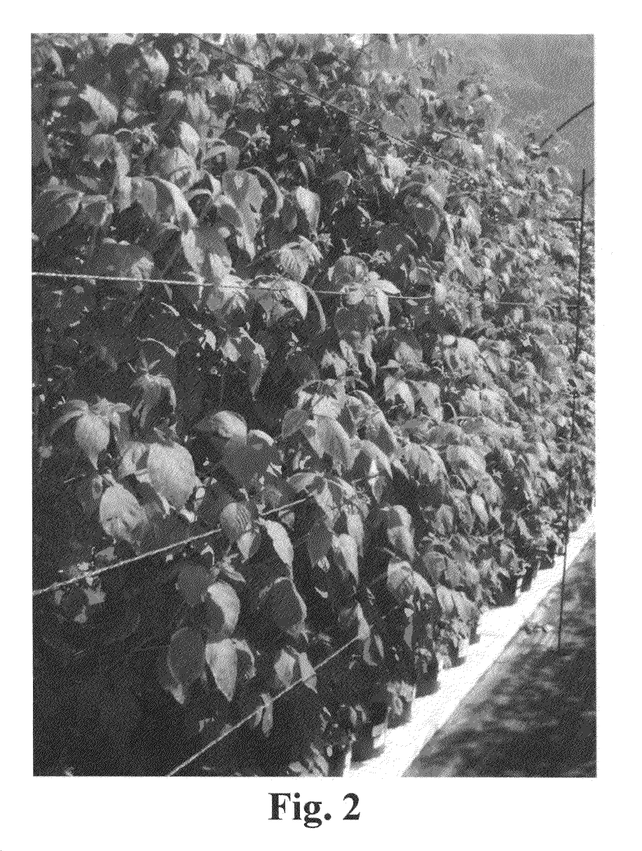

FIG. 3 shows both the ripe and immature fruit size, shape and leaf morphology.

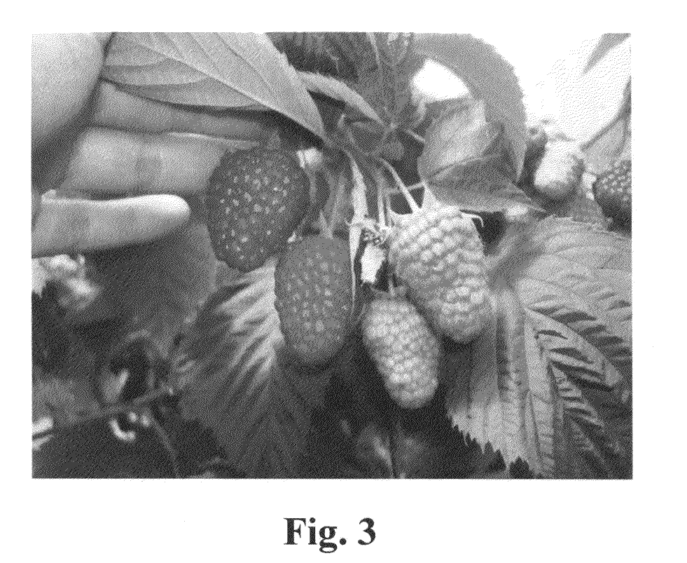

### DETAILED DESCRIPTION

The “Lagorai Plus” is a primocane variety with a very low autumn production, while is highly productive in summer. Therefore the variety is grew as floricane.

The following detailed description set forth the distinctive characteristics of the new variety “Lagorai Plus.” The performance of new cultivar for retention of its distinctive characteristics has been evaluated through successive asexual propagations by root cutting in the Applicant's setting in Trentino (Italy) under controlled conditions of temperature (minimal temperature 15° C. and maximal temperature 28° C.) in greenhouse. The growing was the out of soil using peat as substrate.

The plant assessment was performed for 3 years.
Classification:
   Family.—Rosaceae.
   Botanical.—Rubus idaeus L.
   Common name.—Raspberry.
Parentage Totally unknown.
Plant:
   General habit.—upright canes, very strong vigor, requires support, adapted to medium-high winter chill conditions such as north Italy; very high productivity in summer but not in autumn. Very good roots system.
   Number of current season's shoots.—many to very many.
   Very young shoot.—anthocyanin coloration of apex during rapid growth present;
   Dormant cane colour.—brown.
   Spines.—present; small and uniformly distributed on the plant as typical for red raspberry.
   Leaves.—green on the superior surface and white-tomentose on the inferior surface; odd-pinnate and subdivided in 3-7 ovate and serrate small leafs.
   Bloom.—small and white; the blooming begins in the medium-late season.
Fruit:
   Predominant shape.—conical.
   Weight (mean).—6.8 g.
   Size.—length (mean) 3.50 cm; width (mean) 2.50 cm.
   Colour of ripe fruit.—medium red.
   Main bearing time.—both on previous year's cane in summer and on current year's cane in autumn.
   Time of beginning the fruit ripening on previous season's cane.—medium to late;
   Time of beginning the fruit ripening on current season's cane: late to very late;
   Storage capacity.—good shelf-life high yields of good quality fruit suited to fresh market, good adaptability to frozen market.
Pest and disease resistance: Very strong canes, resistance to pests and diseases; the plants needs few spray treatments for the diseases and pests control. The new cultivar “Lagorai Plus” is similar to the commercial variety “Glen Moy” as shown in Table 1.

TABLE 1
Comparison between “Lagorai Plus” and “Glen Moy”

| Characteristic | “Lagorai Plus” | “Glen Moy” |
|---|---|---|
| Plant | vigorous | |
| Spines | present | absent |
| Fruit | more brilliant; | |
| | firmer; | |
| | late harvest | |
| Fruit ripening time on the | | earlier |
| previous season’s cane | | |

What is claimed:

1. A new and distinct cultivar of raspberry plant as substantially herein shown and described.
